(19) United States
(12) Patent Application Publication
(10) Pub. No.: US 2011/0091916 A1
(43) Pub. Date: Apr. 21, 2011

(54) LUCIFERIN-LUCIFERASE BASED MICRODEVICE FOR BIOSENSING

(75) Inventors: Tao Pan, Shanghai (CN); Zhenghong Sun, Shanghai (CN); Wendy Wang, Shanghai (CN); Xuanbin Liu, Shanghai (CN)

(73) Assignee: Honeywell International Inc., Morristown, NJ (US)

(21) Appl. No.: 12/988,402

(22) PCT Filed: Apr. 21, 2008
(86) PCT No.: PCT/CN08/00811

§ 371 Date: Dec. 23, 2010

(51) Int. Cl. C12Q 1/66; C12M 1/34

(52) U.S. Cl. 435/8; 435/288.1

(57) ABSTRACT

A method and apparatus for determining the concentration of one or more microbes in a sample is provided. This method involves filtering a sample through a filter inside a sample tube to retain the one or more microbes on the filter. The resulting filtrate, which contains or produces adenosine triphosphate, is passed through the sample tube and enters the reporter region. In the reporter region, the adenosine triphosphate in the filtrate comes in contact with a transparent porous matrix, which includes a luciferin-luciferase complex. The adenosine triphosphate interacts with the luciferin-luciferase complex to provide light response, which is measured by a detector. The light response is compared with a calibration curve to determine the total concentration of one or more microbes in a sample.

### BACKGROUND OF THE INVENTION

Bioluminescence is a naturally occurring phenomenon that has been utilized for a number of applications, particularly in molecular biology where the enzyme associated with it have been used as genetic reporters. Bioluminescence is nearly ideal for use as a genetic marker. Typically there is no endogenous luminescent activity in mammalian cells, while the experimentally introduced bioluminescence is nearly instantaneous, sensitive and quantitative. While numerous species exhibit bioluminescence, only a relative few have been characterized and cloned. Of these, only Firefly (Photinus pyralis) luciferase, Renilla luciferase and Aequorin have had much utility. Studies of the molecular components in the mechanism of firefly luciferases producing bioluminescence have shown that the substrate of the enzyme is firefly luciferin, a polyheterocyclic organic acid, D-(−)-2-(6′-hydroxy-2′-benzothiazolyl)-δ²-thiazoline-4-carboxylic acid (hereinafter referred to as “luciferin”).

Firefly luciferase is a monomeric 61 kD enzyme that catalyses the oxidation of luciferin in a two-step process, which yields light at 560 nm. The first step involves the activation of the carboxylate group of luciferin by acylation with the alpha-phosphate of adenosine triphosphate (ATP) in the presence of magnesium to produce luciferyl adenylate with the elimination of inorganic pyrophosphate. In the second step, the luciferyl adenylate is oxidized with molecular oxygen to yield AMP, carbon dioxide and oxyluciferin. The oxyluciferin is generated in an electronically excited state. Upon transition to the ground state the oxyluciferin emits light. The reaction scheme of the reaction hereinafter referred to a luciferin-luciferase reaction is as follows:

Luciferin+ATP+O₂+Mg²⁺+luciferase→oxyluciferin+photons (1)

Since all living cells contain adenosine triphosphate, detection of light in 562 nanometers is indicative of the presence of living cells. The emitted light is recorded and analyzed to determine the concentration of the microbes in the sample. This method has many advantages including, for example, a low detection limit of approximately 10⁻¹⁸ microbes/gram of sample fluid, a short response time of about one minute, and a low reagent and instrument cost.

What is needed is a simple luciferin-luciferase based method and apparatus for determining the concentration of one or more microbes in a sample.

### BRIEF DESCRIPTION OF THE DRAWINGS

Embodiments of the invention may be best understood by referring to the following description and accompanying drawings, which illustrate such embodiments. In the drawings:

FIG. 1 illustrates a cross-sectional view of an exemplary luciferin-luciferase based microdevice for biosensing.

### DETAILED DESCRIPTION

The present invention provides a method and an apparatus for determining the concentrations of one or more microbes in a sample. This method involves filtering a sample through a filter inside a sample tube to retain the one or more microbes on the filter. The resulting filtrate, which contains or produces adenosine triphosphate, may be passed through the sample tube and enters the reporter region. In the reporter region, the adenosine triphosphate in the filtrate comes in contact with a transparent porous matrix, which includes a luciferin-luciferase complex. The adenosine triphosphate interacts with the luciferin-luciferase complex to provide light response, which may be measured by a detector. The detector may be typically a charge-coupled device. The light response may be compared with a calibration curve to determine the total concentration of microbes in a sample. If the specific concentration of a microbe is desired, the sample tube may be cleaned by flushing with buffer or cleaning reagent. Afterward, a second sample may be introduced into the sample tube. This sample may be combined with a reagent that may be specific for the evaluation of desired microbe. The reagent may be, for example, a stimulant, an inhibitor, a stopping reagent, an antibiotic, a nutrient, or a combination thereof. For example, if the reagent is an inhibitor of the specific microbe, that microbe will stop or reduce its ability of producing adenosine triphosphate. As the second sample passes through the filter, the filtrate will have a lower concentration of adenosine triphosphate. This lower concentration of adenosine triphosphate will produce a second light response that may be lower than the original response. ConsequentLy, if the second light response is subtracted from the first light response, the concentration of the specific microbe can be determined. Likewise, this process may be repeated using other regents that arc specific for other microbes. As a result, the concentrations of several microbes may be determined.

As used herein, certain terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 11th Edition, by Sax and Lewis, Van Nostrand Reinhold, New York, N.Y., 1987, and The Merck Index, 11th Edition, Merck & Co., Rahway N.J. 1989.

As used herein, the term “and/or” means any one of the items, any combination of the items, or all of the items with which this term is associated.

As used herein, the singular forms “a,” “an,” and “the” may include plural reference unless the context clearly dictates otherwise. Therefore, for example, a reference to “a formulation” may include a plurality of such formulations, so that a formulation of compound X may include formulations of compound X.

As used herein, the term “about” means a variation of 10 percent of the value specified, for example, about 50 percent carries a variation from 45 to 55 percent. For integer ranges, the term about can include one or two integers greater than and less than a recited integer.

As used herein, the term “antibiotic” refers to a chemotherapeutic agent that inhibits or abolishes the growth of micro-organisms, for example, bacteria, fungi, or protozoa.

As used herein, the term “buffering” refers to a solution that resists changes in the pH.

As used herein, the term “charge-coupled device” refers to a device for forming images electronically, using a layer of silicon that releases electrons when struck by incoming light.

As used herein, the term “inhibitor” refers to an agent that inhibits the growth of microbes.

As used herein, the phrase “in one embodiment” refers a particular feature, structure, or characteristic. However, every embodiment may not necessarily include the particular feature, structure, or characteristic. Further, when a particular feature, structure, or characteristic is described in connection with an embodiment, it is submitted that it is within the knowledge of one skilled in the art to affect such feature, structure, or characteristic in connection with other embodiments whether or not explicitly described.

As used herein, the term “luciferin” refers to any substrate which, upon oxidation by an appropriately chosen luciferase enzyme, produces bioluminescence. Luciferins may be natural or synthetic compounds. Luciferins isolated from different biological species may vary greatly in structure, although in many cases identical structures have been found in widely diverse species.

As used herein, the term “luciferase” refers to an enzyme, which catalyzes the oxidation of a luciferin to oxyluciferin with the generation of light in a bioluminescent reaction such that the oxyluciferin is released from the luciferin-luciferase complex into the reaction medium. The reaction of these luciferases to generate light requires the presence of one or more cofactor such as adenosine triphosphate, nicotinamide adenine dinucleotide/nicotinamide adenine dinucleotide phosphate, or flavin mononucleotide. Examples of such luciferases are those of firefly, bacteria and fungi.

As used herein, the term “luciferin-luciferase complex” refers to any combination of a luciferase with its appropriate luciferin in the presence of all other specific cofactors needed to initiate a bioluminescent reaction, except for the trigger compound as defined herein. There are numerous luciferin-luciferase complexes; each luciferase uses a specific luciferin and co-factors. A luciferase-luciferase complex can be any such combination provided that, on introduction of the trigger compound in its active form, a bioluminescent reaction is initiated and light is generated. A trigger compound is a cofactor needed for the initiation of a luciferase-mediated bioluminescent reaction (other than divalent cations) when added to a luciferin-luciferase complex. Examples of such trigger compound are adenosine triphosphate, nicotinamide adenine dinucleotide/nicotinamide adenine dinucleotide phosphate, or flavin mononucleotide. A trigger compound is chosen to be capable of interaction with the luciferin-luciferase complex in the presence of the appropriately chosen other components (which may include a multivalent cation) to trigger a bioluminescent reaction.

As used herein, the term “microbe” refers to an organism that is too small to be seen by the naked human eye. As used herein, the term “microbe” refers to a bacterium, a fungus, an archaea, or a protist.

As used herein, the term “reporter region” refers to the region on the device that is immobilized with the luciferin-luciferase complex. Once ATP molecules pass through, they will be consumed by luciferin-luciferase and the light will be generated.

As used herein, the term “sample” refers to a material suspected of containing the analyte. The sample can be used directly as obtained from the source or following a pretreatment to modify the character of the sample. The sample can be derived from any biological source, such as a physiological fluid, including, blood, saliva, ocular lens fluid, cerebral spinal fluid, sweat, urine, milk, ascites fluid, raucous, synovial fluid, peritoneal fluid, amniotic fluid or the like. The sample can be pretreated prior to use, such as preparing plasma from blood, diluting viscous fluids, and the like. Methods of treatment can involve filtration, distillation, concentration, inactivation of interfering components, and the addition of reagents. Besides physiological fluids, other liquid samples can be used such as water, food products, and the like for the performance of environmental or food production assays. In addition, a solid material, for example, soil, food, and the like, suspected of containing the analyte can be used as the sample. In some instances it may be beneficial to modify a solid test sample to form a liquid medium or to release the analyte.

As used herein, the term “stimulant” refers to an agent that accelerates the growth of microbes.

As used herein, the term “stopping reagent” refers to an agent that stops, inhibits, or slows the growth of microbes.

As used herein, the term “transparent porous matrix” refers to the ability of the porous matrix to transmit one or more wavelengths of electromagnetic radiation that are generated by the light response provided by the interaction of the filtrate with the luciferin-luciferase complex.

The new method and apparatus can be used to determine enzyme activity, microbe activity, microbe susceptibility to certain reagent, microbe toxicity, disease detection, and the like. Further, this new method and apparatus may find applications in environmental protection, food safety, water quality control, and medical and biological research.

In one embodiment, a method for determining the concentration of one or more microbes in a sample is provided. The method provides (a) filtering a first sample including one or more microbes through a filter inside a sample tube to retain the one or more microbes on the filter and to provide a first filtrate; (b) passing the first filtrate through a reporter region inside the sample tube, wherein the reporter region includes a transparent porous matrix including a luciferin-luciferase complex: (c) detecting a first light response from the reporter region, wherein the light response is provided by the interaction of the filtrate with the luciferin-luciferase complex; (d) comparing the first light response with a calibration curve to determine the concentration of the one or more microbes in the sample; (e) optionally filtering a second sample including one or more microbes and a reagent through the filter inside the sample tube to retain the one or more microbes on the filter and to provide a second filtrate, wherein the reagent is specific for a first microbe and includes a stimulant, an inhibitor, a stopping reagent, an antibiotic, a nutrient, or a combination thereof; (f) optionally passing the second filtrate through the reporter region inside the sample tube; (g) optionally detecting a second light response from the reporter region, wherein the second light response is provided by the interaction of the second filtrate with the luciferin-luciferase complex; (h) optionally subtracting the second light response from the first light response to provide a first microbe specific light response; and (i) optionally comparing the first microbe specific light response with a calibration curve to determine the concentration of the first microbe in the sample, wherein the one or more microbes includes one or more bacteria, one or more fungi, one or more archaea, one or more protists, or a combination thereof.

In one embodiment, the detecting of the first and second light responses is performed with a detector. In one embodiment, the detector includes a camera, a video camera, a silicon photo-cell, or a photo multiplier tube, or a combination thereof.

In one embodiment, the luciferin-luciferase complex is immobilized on or is encapsulated within the transparent porous matrix. In one embodiment, the transparent porous matrix including a luciferin luciferase complex includes a transparent inorganic gel matrix, a transparent organic polymer gel matrix, a transparent hybrid inorganic-organic gel matrix, or a combination thereof. In one embodiment, transparent inorganic gel matrix includes silica gel, borate, TiO₂, Al₂O₃, ZrO₂, or a combination thereof. In one embodiment, the transparent organic polymer gel matrix includes polyvinyl alcohol, polyester, polyimide, polydimethylsiloxane, polymethylmetacrylate, polyolefin, polycarbonate, or a combination thereof.

In one embodiment, the transparent hybrid inorganic-organic gel matrix includes silica gel, borate, TiO₂, Al₂O₃, ZrO₂, polyvinyl alcohol, polyester, polyimide, polydimethylsiloxane, polymethylmetacrylate, polyolefin, polycarbonate, or a combination thereof.

In one embodiment, the sample tube includes an organic polymeric material, an inorganic material, or a combination thereof. In one embodiment, the polymeric material includes a polymethyl methacrylate including polyethylene glycol, polyethylene oxide; polyacrymide, or a combination thereof.

In one embodiment, the inorganic material includes glass, ceramic material, metal, metal alloy, metal oxide, composite metal oxide, or a combination thereof.

In one embodiment, an apparatus in provided. The apparatus includes: (a) a sample tube including: a sample inlet port; a filter inside the sample tube, wherein the sample tube includes an optional reagent inlet between the sample inlet port and the filter or an optional cleaning buffer inlet port connected to an optional valve between the filter and the reporter region; and a reporter region between the filter and an outlet port, wherein the reporter region includes a transparent porous matrix including a luciferin-luciferase complex; (b) a detector coupled to the detector to detect one or more light responses from the reporter region, wherein the one or more light responses are provided by the interaction of one or more filtrates with the luciferin-luciferase complex; and (c) an analyzer coupled to the reporter region to determine the concentration of the one or more microbes in the sample, wherein the one or more microbes includes one or more bacteria, one or more fungi, one or more archaea, one or more protists, of a combination thereof.

In one embodiment, an apparatus is provided. The apparatus includes: (a) a sample tube including: a sample inlet port, a filter inside the sample tube; and a reporter region between the filter and an outlet port, wherein the reporter region includes a transparent porous matrix including a luciferin-luciferase complex; (b) a detector coupled to the reporter region to detect one or more light responses from the reporter region, wherein the one or more light responses are provided by the interaction of one or more filtrates with the luciferin-luciferase complex; and (c) an analyzer coupled to the detector to determine the concentration of the one or more microbes in the sample.

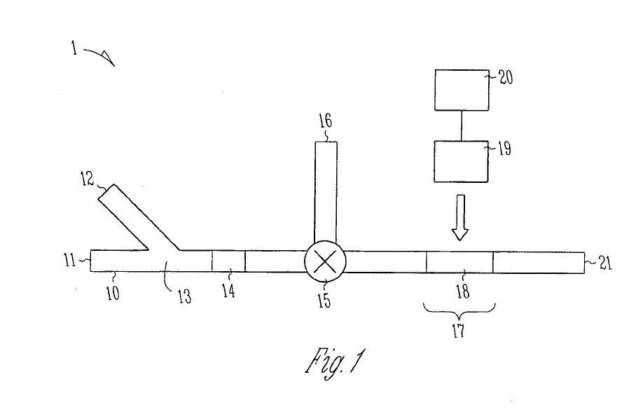

FIG. 1 is a cross-sectional view of an exemplary luciferin-luciferase based microdevice (1) for biosensing.

In one embodiment, the microdevice (1) includes a sample tube (10), a sample reservoir (11), a reagent reservoir (12), a mixing area (13), a filter (14), a valve (15), a cleaning buffer inlet reservoir (16), a reporter region (17), a transparent porous matrix (18), a detector (19), an analyzer (20), and an outlet reservoir (21). The operator opens the valve (15) to connect the sample reservoir (11) with the outlet reservoir (21). The sample and the reagent are pumped into the mixing area (13), combined, and the mixed sample pumped through the filter (14). The filter (14) will retain the microbes and other large debris and allow the small molecules (e.g., adenosine triphosphate) dissolved in the sample fluid to pass through the filter (14). The fluid will pass through the valve (15) and enter the reporter region (17). Once in the reporter region (17), the fluid will enter the transparent porous matrix (18) that contains the luciferin-luciferase complex. The luciferin-luciferase complex may be immobilized on or may be encapsulated inside the transparent porous matrix.

As discussed above, the adenosine triphosphate reacts with a luciferin-luciferase complex, in the presence of oxygen and magnesium ions (Mg⁺²) to produce visible light at a wavelength of 562 nanometers according to equation (1). The visible light of 562 nanometers may be received by the detector (19), converted into a signal that may be sent to the analyzer (20). The analyzer (20) compares the signal with a calibration curve to determine the concentration of the microbe in the sample. When the data analysis is complete, the operator may open the valve (15), pump cleaning buffer from the cleaning buffer reservoir (16) through the porous matrix (18) and regenerate the luciferin-luciferase to complex immobilized on or encapsulated inside the transparent porous matrix (18). Next, the operator may replace the sample reservoir (11) and reagent reservoir (12) with waste reservoirs (not shown), open the valve (15), and pump cleaning buffer from the cleaning buffer reservoir (16) to the waste reservoirs thereby cleaning the filter of any retained microbes and other large debris. The microdevice (1) is ready for the next sample. Although not shown in the FIGURE, the microdevice (1) may be provided with a temperature-regulating mechanism for keeping the fluids at a predetermined temperature and with a mixing mechanism for stirring the mixture in the mixing area (13).

Any microbe-containing liquid may be used as the sample. A culture solution obtained, for example, by culturing bacteria in a medium, or a clinical sample such as bacteria-containing urine and blood, or the like can be used as the sample.

In some embodiments, the reagent reservoir (12), a mixing area (13), a valve (15), and a cleaning buffer reservoir (16) are optional.

Suitable microbes may include, for example, one or more bacteria, one or more fungi, one or more archaea, one or more protists, of a combination thereof.

### Detection Devices

In another embodiment, the detecting of the first and second light responses may be performed with a charge-coupled device. Other suitable devices may include, for example, a camera, a video camera, a silicon photo-cell, a photo multiply tube (PMT), and the like, or combinations thereof.

Suitable charge-coupled devices include, for example, CoolSNAP EZ available from Photometrics, Pleasanton, Calif., USA.

### Immobilized Luciferin-Luciferase Complex

In one embodiment, the luciferin-luciferase complex may be immobilized on the transparent porous matrix. Several methods may be used including, for example, immobilizing luciferase onto porous matrix (with amine treated surfaces) through diazotization; directly immobilizing luciferase onto porous matrix (with amine treated surfaces) through Schiff base reaction; or directly immobilizing luciferase onto porous matrix (with carboxyl, aldehyde groups) through Schiff base reaction. As to the luciferin, two methods may be used, including, for example, adding luciferin into a solution and allow the solution to flow through reporter region with ATP; and immobilizing luciferin onto porous matrix using, for example, the luciferin phenolic group.

In another embodiment, the luciferin-luciferase complex may be encapsulated within the transparent porous matrix. To encapsulate the luciferin-luciferase complex within the matrix, two methods, for example, may be used. In the first method, cells or microbes that express luciferin-luciferase are grown and encapsulate within a porous matrix using, for example, sol-gel or other technologies. In the second method, luciferase may be incorporated into a porous matrix with a pore size smaller than the size of luciferase. In each of the above methods, luciferin may be added, for example, by (1) adding luciferin into solution and allow the luciferin-containing solution to flow through reporter region with ATP; (2) immobilizing the luciferin onto porous matrix using the phenolic group of luciferin; and (3) producing luciferin with cells or microbes that are encapsulated within the matrix.

In another embodiment, the transparent porous matrix including a luciferin-luciferase complex includes a transparent inorganic gel matrix, a transparent organic polymer gel matrix, a transparent hybrid inorganic-organic gel matrix, or a combination thereof.

In one embodiment, the transparent inorganic gel matrix includes silica gel, borate, TiO₂, Al₂O₃, ZrO₂, or a combination thereof.

In one embodiment, the transparent organic polymer gel matrix includes polyvinyl alcohol, polyester, polyimide, polydimethylsiloxane, polymethylmetacrylate, polyolefin, polycarbonate, or a combination thereof.

In one embodiment, the composite gel matrix includes a combination of above inorganic and organic polymer gel matrix thereof.

Suitable transparent inorganic gel matrices and transparent hybrid inorganic-organic gel matrices are disclosed in Schubert et al., Synthesis of Inorganic Materials, 2^{nd} Edition, Wiley-VCH, Weinheim, Germany, 2005.

Suitable transparent organic polymer gel matrices may include, for example, water-insoluble materials such as cellulose acetates, cellulose propionates, cellulose butyrates, cellulose acetate butyrates, cellulose nitrates, polyacrylates, polyethylene terephthalates, polyethylenes, polymethacrylates, polystyrenes, polypropylenes, polycarbonates, polyvinyl acetals, polyimides, polysiloxanes, polyurethanes, polysaccharides, polyvinylalcohols, polyacrylamides, copolymers thereof, or combinations thereof.

### Sample Tube

In one embodiment, the sample tube includes a polymeric material or an inorganic material, or other biocompatible or biologically unreactive material. The sample tube may be, for example, entirely made of one material, or be made of different sections, each of which may be composed of a different material.

In one embodiment, the sample tube may be composed of one material. In another embodiment, the sample tube may be composed of two or more materials in different sections.

In one embodiment, the sample tube may be made entirely out of glass or a polymer. In another embodiment, the sample tube may be made of metal with a transparent reporter region made of transparent material such as glass or a polymer. In certain embodiments in which the sample tube may be made of more than one material (e.g., glass and metal), a sealant may be used to connect the materials. In one embodiment, a silicon sealant may be used such as Room Temperature Vulcanizing (RTV) silicone rubber sealants.

The sample tube may be chosen to provide appropriate light-transmitting characteristics. For instance, the sample tube may be a functionalized or non-functionalized glass or any one of a wide variety of polymers. Other substrate materials will be readily apparent to those of skill in the art upon review of this disclosure.

Suitable polymeric materials for the sample tube may include, for example, transparent organic polymers that are homopolymers or copolymers, naturally occurring or synthetic, crosslinked or uncrosslinked. Specific polymers of interest include, but are not limited to, polyolefins such as polypropylene, polyimides, polycarbonates, polyesters, polyamides, polyethers, polyurethanes, polyfluorocarbons, polystyrenes, poly(acrylonitrile-butadiene-styrene)(ABS), acrylate and acrylic acid polymers such as polymethyl methacrylate, and other substituted and unsubstituted polyolefins, and copolymers thereof.

The sample tube may also be fabricated from a “composite,” i.e., a composition comprised of unlike materials. The composite may be a block composite, e.g., an A-B-A block composite, an A-B-C block composite, or the like. The interior surface of the sample tube may be chemically modified to provide desirable chemical or physical properties, e.g., to reduce adsorption of molecular moieties to the interior walls of the sample tube, and to reduce electro osmotic flow. For example, the interior surface of the sample tube may be coated with or functionalized to contain electrically neutral molecular species, zwiterrionic groups, hydrophilic or hydrophobic oligomers or polymers, etc. With polyimides, polyamides, and polyolefins having reactive sites or functional groups such as carboxyl, hydroxyl, amino and haloalkyl groups (e.g., polyvinyl alcohol, polyhydroxystyrene, polyacrylic acid, polyacrylonitrile, etc.), or with polymers that can be modified so as to contain such reactive sites or functional groups, it may be possible to chemically bond groups to the surface that can provide a variety of desirable surface properties. The interior surface of the sample tube may also be advantageously modified using surfactants (e.g., polyethylene oxide triblock copolymers such as those available under the tradename “Pluronic,” polyoxyethylene sorbitan, or “TWEEN”), natural polymers (e.g., bovine serum albumin), or other moieties that provide the desired surface characteristics, particularly in reducing the adsorption of biomolecules such as nucleic acids or proteins.

Preferably, the polymeric material in the sample tube may include, for example, a polymethyl methacrylate including polyethylene glycol, polyethylene oxide; polyacrymide, and the like, or a combination thereof. In one embodiment, the polymethyl methacrylate including polyethylene glycol, polyethylene oxide; polyacrymide, and the like, or a combination thereof may be, for example, surface modified.

Suitable inorganic materials for the sample tube may include, for example, glass, ceramic material, metal, metal alloy, metal oxide, composite metal oxide, or a combination thereof.

Suitable ceramic materials may include, for example, SiO₂, ZrO₂, TiO₂, Al₂O₃, ZnO, and the like, or a combination thereof.

Suitable metals may include, for example, Al, Mg, Zn. Pd, Pt, Ni, Co, Rh, Ir, Fe, Ru, Au, Ag, Cu, and the like, or a combination thereof.

Suitable alloys may include, for example, stainless steel, duralumin, silumin, bronze, brass, and the like, or a combination thereof.

Another possible solution for this sample tube may be to use the any combination of above materials.

### Operating Conditions

The apparatus for determining the concentration of one or more microbes in a sample may be operated at, for example, any desired temperature, pressure, and flow rate that may be suitable for the desired microbe.

In an exemplary embodiment, the apparatus may be operated at a temperature from about 0° C. to about 100° C., typically from about room temperature to about 60° C., and more typically from about 35° C. to about 40° C. However, in certain embodiments wherein the desired microbe is a microbe suited for high temperature environments, higher temperatures may be used.

The flow rate of the sample inside the sample tube may be at any desired rate. In an exemplary embodiment, the flow rate may be from about 0.000005 standard cubic centimeters per minute (sccm) to about 10 sccm, preferably from about 0.00005 sccm to about 0.1 sccm, more preferably from about 0.0005 sccm to about 0.01 sccm.

The pressure inside the sample tube may be at any desired pressure.

All patents and publications referenced or mentioned herein are indicative of the levels of skill of those skilled in the art to which the invention pertains, and each such referenced patent or publication is hereby incorporated by reference to the same extent as if it had been incorporated by reference in its entirety individually or set forth herein in its entirety. Applicant reserves the right to physically incorporate into this specification any and all materials and information from any such cited patents or publications.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and that they are not necessarily restricted to the orders of steps indicated herein or in the claims.

What is claimed is:

1. A method for determining the concentration of one or more microbes in a sample, comprising the steps of:
(a) filtering a first sample comprising one or more microbes through a filter inside a sample tube to retain the one or more microbes on the filter and to provide a first filtrate;
(b) passing the first filtrate through a reporter region inside the sample tube, wherein the reporter region comprises a transparent porous matrix comprising a luciferin-luciferase complex:
(c) detecting a first light response from the reporter region, wherein the light response is provided by the interaction of the filtrate with the luciferin-luciferase complex;
(d) comparing the first light response with a calibration curve to determine the concentration of the one or more microbes in the sample;
(e) optionally filtering a second sample comprising one or more microbes and a reagent through the filter inside the sample tube to retain the one or more microbes on the filter and to provide a second filtrate, wherein the reagent is specific for a first microbe and comprises a stimulant, an inhibitor, a stopping reagent, an antibiotic, a nutrient, or a combination thereof;
(f) optionally passing the second filtrate through the reporter region inside the sample tube;
(g) optionally detecting a second light response from the reporter region, wherein the second light response is provided by the interaction of the second filtrate with the luciferin-luciferase complex;
(h) optionally subtracting the second light response from the first light response to provide a first microbe specific light response; and
(i) optionally comparing the first microbe specific light response with a calibration curve to determine the concentration of the first microbe in the sample,
wherein the one or more microbes comprises one or more bacteria, one or more fungi, one or more archaea, one or more protists, or a combination thereof.

2. The method of claim 1, wherein the detecting of the first and second light responses is performed with a detector.

3. The method of claim 2, wherein the detector comprises a camera, a video camera, a silicon photo-cell, or a photo multiplier tube, or a combination thereof.

4. The method of claim 1, wherein the luciferin-luciferase complex is immobilized on or is encapsulated within the transparent porous matrix.

5. The method of claim 1, wherein the transparent porous matrix comprising a luciferin-luciferase complex comprises a transparent inorganic gel matrix, a transparent organic polymer gel matrix, a transparent hybrid inorganic-organic gel matrix, or a combination thereof.

6. The method of claim 5, wherein the transparent inorganic gel matrix comprises silica gel, borate, TiO₂, Al₂O₃, ZrO₂, or a combination thereof.

7. The method of claim 5, wherein the transparent organic polymer gel matrix comprises polyvinyl alcohol, polyester, polyimide, polydimethylsiloxane, polymethylmetacrylate, polyolefin, polycarbonate, or a combination thereof.

8. The method of claim 5, wherein the transparent hybrid inorganic-organic gel matrix comprises silica gel, borate, TiO₂, Al₂O₃, ZrO₂, polyvinyl alcohol, polyester, polyimide, polydimethylsiloxane, polymethylmetacrylate, polyolefin, polycarbonate, or a combination thereof.

9. The method of claim 1, wherein the sample tube comprises an organic polymeric material, an inorganic material, or a combination thereof.

10. The method of claim 9, wherein the polymeric material comprises a polymethyl methacrylate comprising polyethylene glycol, polyethylene oxide; polyacrymide, or a combination thereof.

11. The method of claim 9, wherein the inorganic material comprises glass, ceramic material, metal, metal alloy, metal oxide, composite metal oxide, or a combination thereof.

12. Apparatus, comprising:
(a) a sample tube comprising:
a sample inlet port;
a filter inside the sample tube, wherein the sample tube comprises an optional reagent inlet between the sample inlet port and the filter or an optional cleaning buffer inlet port connected to an optional valve between the filter and the reporter region; and
a reporter region between the filter and an outlet port, wherein the reporter region comprises a transparent porous matrix comprising a luciferin-luciferase complex;
(b) a detector coupled to the reporter region to detect one or more light responses from the reporter region, wherein the one or more light responses are provided by the interaction of one or more filtrates with the luciferin-luciferase complex; and
(c) an analyzer coupled to the detector to determine the concentration of the one or more microbes in the sample, wherein the one or more microbes comprises one or more bacteria, one or more fungi, one or more archaea, one or more protists, of a combination thereof.

13. The apparatus of claim 12, wherein the detector comprises a camera, a video camera, a silicon photo-cell, or a photo multiplier tube, or a combination thereof.

14. The apparatus of claim 12, wherein the luciferin-luciferase complex is immobilized on or is encapsulated inside the transparent porous matrix.

15. The apparatus of claim 12, wherein the transparent porous matrix comprising a luciferin-luciferase complex comprises a transparent inorganic gel matrix, a transparent organic polymer gel matrix, a transparent hybrid inorganic-organic gel matrix, or a combination thereof.

16. The apparatus of claim 15, wherein the transparent inorganic gel matrix comprises silica gel, borate, TiO₂, Al₂O₃, ZrO₂, or a combination thereof.

17. The apparatus of claim 15, wherein the transparent organic polymer gel matrix comprises polyvinyl alcohol, polyester, polyimide, polydimethylsiloxane, polymethylmetacrylate, polyolefin, polycarbonate, or a combination thereof.

18. The apparatus of claim 15, wherein the transparent hybrid inorganic-gel matrix comprises silica gel, borate, TiO₂, Al₂O₃, ZrO₂, polyvinyl alcohol, polyester, polyimide, polydimethylsiloxane, polymethylmetacrylate, polyolefin, polycarbonate, or a combination thereof.

19. The apparatus of claim 12, wherein the sample tube comprises an organic polymeric material, an inorganic material, or a combination thereof.

20. Apparatus, comprising:
(a) a sample tube comprising:
a sample inlet port;
a filter inside the sample tube; and
a reporter region between the filter and an outlet port, wherein the reporter region comprises a transparent porous matrix comprising a luciferin-luciferase complex;
(b) a detector coupled to the reporter region to detect one or more light responses from the reporter region, wherein the one or more light responses are provided by the interaction of one or more filtrates with the luciferin-luciferase complex; and
(c) an analyzer coupled to the detector to determine the concentration of the one or more microbes in the sample.
